(19) United States
(12) Patent Application Publication
(10) Pub. No.: US 2024/0100212 A1
(43) Pub. Date: Mar. 28, 2024

(54) REVERSIBLY PROTECTED COLORANTS AND METHODS OF USE

(71) Applicant: Kinnos Inc., Brooklyn, NY (US)

(72) Inventors: Kara Ng, Brooklyn, NY (US); Matthew Jurow, Brooklyn, NY (US); Pau Oliveres, Brooklyn, NY (US); Dorothy Jones, Brooklyn, NY (US)

(21) Appl. No.: 18/375,604

(22) Filed: Oct. 2, 2023

Related U.S. Application Data
(63) Continuation of application No. PCT/US2022/026302, filed on Apr. 26, 2022.
(60) Provisional application No. 63/180,262, filed on Apr. 27, 2021.

(51) Int. Cl. A61L 2/28; A01N 25/04; A01N 37/02; A01P 1/00; A61L 2/18; B01J 13/10; C09B 67/02

(52) U.S. Cl. CPC A61L 2/28; A01N 25/04; A01N 37/02; A01P 1/00; A61L 2/18; B01J 13/10; C09B 67/0097

(57) ABSTRACT

The invention provides a reversibly protected colorant or a composition thereof which when mixed with a disinfectant solution imparts color to the disinfectant solution allowing for temporary visualization of a disinfectant, and methods for use in the disinfection, decontamination, and/or cleaning of surfaces, objects. The reversibly protected colorant or composition thereof is added to a disinfectant solution or disinfectant article (e.g., disinfectant wipe, microfiber, rag, towel, cloth, spun-woven wipes, nonwoven wipes, etc.), wherein the reversibly protected colorant or a composition thereof imparts color to the disinfectant solution resulting in a colored disinfectant solution until such protection is removed, thereby releasing the colorant and exposing the colorant to an agent and/or oxidizing conditions that cause the colorant to permanently lose its color. The invention also provides methods of using the colored disinfectant solutions for visually indicating surface disinfection and the method of visually indicating surface disinfection.

### RELATED APPLICATIONS

This application is a continuation of International Application No. PCT/US22/26302, which designated the United States and was filed on Apr. 26, 2022, published in English, which claims the benefit of U.S. Provisional Application No. 63/180,262, filed on Apr. 27, 2021. The entire teachings of the above applications are incorporated herein by reference.

### BACKGROUND OF INVENTION

Surface disinfection is essential to preventing infectious disease. Surface disinfection relies on proper technique and complete application to effectively eliminate transmission of infection.

Current methods for surface disinfection are prone to human error. The majority of disinfectants and soaps are transparent or only slightly hued, making it difficult for users to visualize coverage to ensure complete disinfection. In addition, current disinfectants and sanitizers do not provide a real-time feedback system to ensure the proper length of time for application, which results in poor compliance with surface disinfectant contact time requirements, often ranging up to 10 minutes, and insufficient handwashing durations (cdc.gov/mmwr/PDF/rr/rr5116.pdf).

As a result, less than 50% of hospital surfaces are properly cleaned (doi:10.1086/524329) while rates of adherence to handwashing average less than 40% among hospital staff (cdc.gov/mmwr/PDF/rr/rr5116.pdf). Poor cleaning and handwashing technique have dire consequences: in the United States alone, 1 out of every 31 patients will contract a healthcare-associated infection (HAI), leading to over 99,000 patient deaths and $45B in direct costs to the healthcare system annually. There thus exists a need for improved methods and compositions around surface disinfectants and hand soaps and sanitizers to ensure proper use and reduce infection.

Commercially available products attempt to improve cleaning and handwashing compliance but do not fully address the core issue of human error. For example: Glo Germ™ and 3M Clean Trace™ can be used to check missed spots or remaining bioburden after cleaning but are retrospective interventions and do not provide real-time feedback. Monitoring systems from Biovigil or Hygreen are physical devices that remind healthcare workers to wash their hands upon entering or leaving a patient room but can be cumbersome to implement and do not address the issue of poor handwashing technique.

The addition of dyes that conveys a color to the solution to which it is added is a potential method for improving the visibility of disinfecting agents. For example, U.S. Pat. No. 4,308,625 to Kitko and U.S. Pat. No. 4,229,410 to Kosti both teach the use of dyes in combination with sanitizing agents for the purpose of providing transient coloration of toilet bowl water with each flush. Both U.S. Pat. No. 5,110,492 to Casey and U.S. Pat. Application 2014/0057987 disclose the combination of a cleaning solution with a pH dye that changes in color in response to being dispensed from an airtight container. U.S. Pat. No. 10,052,398 to Kang teaches the addition of an oxidizable dye that colorizes a disinfectant and fades to clear over time in reaction with an oxidizing agent (e.g., sodium hypochlorite).

Methods for the composition of color changing hand soaps and sanitizers have previously been disclosed. Cleansing compositions with colorants that change or fade in color through a chemical reaction have been well described. U.S. Pat. No. 7,858,568 to MacDonald describes a color change composition that is stable in single phase and produces an observable color change to indicate the thoroughness of hand cleaning. This patent discloses a liquid cleaning formulation that incorporates a redox or pH sensitive dye that reacts either with a reducing agent or from a pH change generated by a catalyst. U.S. Pat. Application 2006/0257439 to Sabnis, U.S. Pat. Application 2008/0223413 to Radford, and U.S. Pat. No. 8,236,744 to Boyke all describe soaps and cleansing compositions that include a pH sensitive indicator dye to produce a color change upon handwashing. The color change observed in pH sensitive indicators are reversible, rendering these solutions inadequate for the described problem.

Methods of encapsulating colorants have also been disclosed, generally with the intention of providing permanent coloration to a surface. U.S. Pat. No. 5,484,475 to Breton teaches a process for preparing an ink composition with micelles comprising an ethoxylated alcohol. U.S. Pat. No. 6,841,591 to Vincent describes a method for stabilizing a dye with a transparent polymeric matrix to form an encapsulated particle and enable the use of hydrophobic colorants in a hydrophilic vehicle. U.S. Pat. No. 9,555,147 to Song describes an ink that can change from colorless to colored via an encapsulation matrix that is insoluble in water but soluble in organic solvents. Both U.S. Pat. No. 8,846,404 to Odom and U.S. Pat. No. 9,245,202 to Boday teach a visual indicator of mechanical damage in which rupturing of capsules allows a reaction that elicits color change. Other examples of colorants that are contained within microcapsules are taught by U.S. Pat. No. 9,675,533 to Zhu and European Pat. No. 2293761 to Kvitnitsky.

Compositions with colorants that change or fade in color through mechanical force or temperature change have also been disclosed. Both U.S. Pat. No. 8,067,350 B2 to Wenzel and U.S. Pat. Application 2006/60287215 Al to McDonald describe a cleaning composition prepared with a single or multiple thermochromic dyes that change color in response to temperature changes from friction or warm water during handwashing. U.S. Pat. Application 2006/0040835 A1 to Newkirk discloses a cleansing composition that incorporates colorant particles suspended in surfactant solution that change color in response to shear forces. U.S. Pat. No. 8,680,032 to Lachmann describes microencapsulated colorant granules in which, during hand scrubbing, the shell encapsulation decomposes and releases colorants to induce a color change of the foam. In these technologies the residual colorant does not lose its color but must be physically removed from the local environment of the remaining dye, generally with running water.

There is thus a need for compositions and methods to improve surface disinfection. In addition, there remains a need for a real-time indicator of proper friction and sufficient elapsed time. Whereas previous color changing techniques are designed to impart a color to the soap foam during the handwashing process, this visible color must be rinsed off with water, which precludes their use in rinse-free sanitizers or lotions. A need currently exists for a colorant that can be used in cleansing or surface disinfection that provides visible color during use, but effectively and permanently fades without requiring rinsing with water.

### DESCRIPTION OF FIGURES

The patent or application file contains at least one drawing executed in color. Copies of this patent or patent application publication with color drawings will be provided by the Office upon request and payment of the necessary fee.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts gelatin-chitosan microcapsules loaded with a colorant. Specifically, gelatin B (3.09 g) and chitosan (0.02 g) were dissolved in 1% w/w acetic acid. Thymolphthalein (0.2 gm) was dispersed in Tween 80 (5 gm), and then mixed into chitosan (20 mL) under stirring at 50° C. for 30 minutes. After, gelatin solution (20 mL) was added to dye chitosan mixture at 1 mL/min using a syringe and allowed to stir for 30 minutes. pH of resulting colloid was adjusted to 5.50 by slow, dropwise addition of 1M NaOH and stirred for 4 hours to induce coacervation. Liquid coacervate was cooled gradually to RT then incubated in ice bath under constant stirring for 1 hour. Formaldehyde (2.5% v/v) was added dropwise into mixture and stirred for 30 minutes to induce cross linking. Crude coacervate mixture was washed and centrifuged 3× with ethanol and 1× with cold water (1000 rpm, 5 minutes, 10 10° C.). Washed microcapsules were freeze dried overnight.

FIG. 2 depicts a sample of an encapsulated colorant and a sample of the same colorant in its native state in water before and after addition of KOH. Specifically, un-encapsulated and encapsulated thymolpthalein (25 mg) in 5 mL deionized water before (left) and 1 hour after (right) addition of 500 μL 40 mM KOH (final pH=11.2).

FIG. 3 depicts microcapsules of a colorant after synthesis and purification. Specifically, poly(vinyl alcohol) (PVA, 30-70K MW) was added dropwise to deionized water (100 mL) under mechanical stirring at 85° C. for three hours to prepare 5% PVA solution. Monomer solution was prepared by dissolving benzoyl peroxide (0.5% wt., 0.141 g) in 30 mL methyl methacrylate; dye-monomer solution was prepared by adding 3 mL rhodamine B stock (50 mM in ethanol) under mechanical stirring. After cooling to room temperature, PVA solution was stirred at 700 RPM and the dye-monomer mixture was added dropwise to PVA over 10 minutes, the mixture was heated to 70° C. under mechanical stirring for three hours. After, the mixture was cooled to room temperature. Crude mixture was washed and centrifuged with cold, distilled water (1000 rpm, 3 minutes). Washed microcapsules were refrigerated. Microcapsule size range: 0.2-1.0 μm.

FIG. 4A through FIG. 4C demonstrates depletion of optical absorption of an encapsulated colorant, e.g., methyl-methacrylate encapsulated rhodamine B, and its response to mechanical stress and subsequent exposure to a fading agent. FIG. 4A shows rhodamine microcapsules deposited and dried on a surface. FIG. 4B shows microcapsules after they were deposited and rubbed onto surface. FIG. 4C shows rhodamine microcapsules after they were deposited and rubbed onto surface in the presence of a fading agent.

### DETAILED DESCRIPTION OF THE INVENTION

The invention provides reversibly protected species. A reversibly protected species is a reversibly protected colorant and/or a reversibly protected fading agent as described herein. The invention also provides compositions. The composition can comprise the reversibly protected colorant. In embodiments, the reversibly protected colorant is free of any surfactants, hydrotropes, thickening agents, or alkaline builders. In some embodiments, the composition consists of the reversibly protected colorant.

In embodiments, the invention provides a composition comprising a reversibly protected fading agent. In embodiments, the reversibly protected fading agent is free of any surfactants, hydrotropes, thickening agents, and/or alkaline builders. In some embodiments, the composition consists of the reversibly protected fading agent.

The invention provides a reversibly protected colorant or a composition thereof and methods for use in the disinfection, decontamination, and/or cleaning of surfaces, objects. The reversibly protected colorant or composition thereof is added to a disinfectant solution or disinfectant article (e.g., disinfectant wipe, microfiber, rag, towel, cloth, spun-woven wipes, nonwoven wipes, etc.), wherein the reversibly protected colorant or a composition thereof imparts color to the disinfectant solution resulting in a colored disinfectant solution until such protection is removed, thereby releasing the colorant and exposing the colorant to an agent or oxidizing conditions that cause the colorant to permanently lose its color. The invention also provides methods of using the colored disinfectant solutions for visually indicating surface disinfection and the method of visually indicating surface disinfection.

The reversibly protected colorant or composition thereof of the invention can be in a solid form. In certain embodiments, compositions of the invention may be compressed to pills or tablets to facilitate dissolving at a controlled (e.g., slower) rate and to make transportation, storage, and/or implementation easier or suited for specific applications.

The reversibly protected colorant or composition thereof of the invention can be an aqueous composition, which can be added to a disinfectant.

The reversibly protected colorant or composition thereof are used to enhance the applicability of conventional disinfectants, including, but not limited to, bleaches, chlorines and chlorine compounds, quaternary ammonium compounds, alcohols, hydrogen peroxides, accelerated hydrogen peroxides, acids, formaldehydes, glutaraldehydes, iodophors, orthophthalaldehydes, peracetic acids, phenolics, or combinations of disinfectants, by ensuring complete and correct application. The reversibly protected colorant or composition thereof is easily deployable on site and can be added to disinfectants at point-of-use or at point-of-manufacture, for a variety of use cases and industries including, but not limited to, healthcare, dental, transit, transportation, food services, industrial, laboratory, commercial, consumer, hospitality, entertainment, real estate, and more.

The invention is primarily intended for use in surface disinfection. In some embodiments, the colored disinfectant solution comprises a reversibly protected colorant and a disinfectant solution, as defined herein. In other embodiments, the colored disinfectant solution further comprises a fading agent as defined herein. In some embodiments, the fading agent is a reversibly protected fading agent.

The reversibly protected colorant may be combined with a disinfectant solution or disinfectant article and stored prior to use. In some embodiments, the reversibly protected colorant is a solid composition and can be diluted in an aqueous solution, such as water. In some embodiments, the aqueous solution is the disinfectant solution. In some embodiments, both the reversibly protected colorant and disinfectant are solid compositions, which can be diluted with solvents, often water, and mixed together. In some embodiments, the colorant is a solid or liquid composition and can be diluted in solvents, including, but not limited to, water, alcohol, and other solutions.

In embodiments, the reversibly protected colorant can be mixed with a disinfectant solution immediately prior to use. As used herein, “immediately prior to use” refers to a period of time of a second to several hours prior to application to a surface or object to be disinfected. In embodiments, the reversibly protected colorant is combined with the disinfectant at the point of use, for example upon application to a surface or object to be disinfected.

In embodiments, the disinfectant is a disinfectant article and the reversibly protected colorant is applied to the wipe prior to using the wipe to clean a surface or object. The disinfectant article can include, but is not limited to, a wipe, microfiber, rag, towel, cloth, spun-woven wipes, nonwoven wipes. In embodiments, a fading agent is also applied to the disinfectant article. In some embodiments, the fading agent is a reversibly protected fading agent.

In embodiments, the disinfectant and the reversibly protected colorant is applied to a wipe prior to using the wipe to clean a surface or object.

In any of the embodiments described herein, the colored disinfectant solution can be applied to the surface or object to be disinfected as a spray or film.

In one embodiment, the reversibly protected colorant is stable in the solution comprising the disinfectant and is sufficient to color the disinfectant solution. Upon application to the surface or object to be disinfected, the color of the disinfectant solution clearly marks where the disinfectant solution has or has not been applied. The reversibly protected colorant is configured to release the colorant from its protection after exposure to chemical or mechanical action on the colored disinfectant solution. For example, the application of mechanical action (e.g., wiping or rubbing the surface or object with the colored disinfectant solution) depletes the imparted protection allowing for exposure of the colorant, which in turn removes the color from surface or object after a period of time.

In embodiments, the mechanical action, such as rubbing, spreads the colored disinfectant solution and facilitates the release of the colorant from its protection as shear forces resulting from the mechanical agitation of the solution on the surface or object can remove the protection or release the colorant from its protection or, alternatively, cause adequate abrasion of the colorant particles, which in turn exposes the colorant and causes the color of the disinfectant solution to fade to clear over a period of time. Removal of the protection and release of the colorant exposes the colorant to the disinfectant solution, which itself can cause the colorant to lose its color, or exposes the colorant to an external source, such as exposure of the colorant to air, light, solvents, oxidizers, catalysts, or pH conditions. In embodiments, removal of the protection and release of the colorant exposes the color to the disinfectant solution, which itself can cause the colorant to lose its color. This loss of color by the disinfectant solution is observable to the user. In some embodiments, the color may change to a different color or a series of colors before permanently fading to clear.

In an embodiment, a reversibly protected colorant and a fading agent are added to and are stable in the solution comprising the disinfectant and the reversibly protected colorant is sufficient to color the disinfectant solution. The reversibly protected colorant is configured to release the colorant from its protection after exposure to chemical or mechanical action on the disinfection solution. For example, the application of mechanical action depletes the imparted protection releasing the colorant, which exposes the colorant to the fading agent, wherein the fading agent degrades the colorant and causes the color of the disinfectant solution to fade after a period of time.

In another embodiment, a reversibly protected colorant and reversibly protected fading agent are added to and are stable in the solution comprising the disinfectant and the reversibly protected colorant is sufficient to color the disinfectant solution. The reversibly protected colorant and reversibly protected fading agent are configured to release the colorant and fading agent from their protection after exposure to chemical or mechanical action on the colored disinfection solution. For example, the application of mechanical action depletes the imparted protection releasing both the colorant and fading agent, which exposes the colorant to the fading agent, wherein the fading agent degrades the colorant and causes the color of the disinfectant solution to fade after a period of time.

In another embodiment, a reversibly protected fading agent and a colorant are added to and are stable in the solution comprising the disinfectant, wherein the colorant imparts a color to the disinfectant solution. The reversibly protected fading agent is configured to release fading agent from its protection after exposure to chemical or mechanical action on the colored disinfection solution. For example, the application of mechanical action depletes the imparted protection the fading agent, releasing the fading agent into the solution. The disinfectant solution remains colored for a period of time, enabling users to visualize surface coverage before the fading agent degrades the colorant and causes the color of the disinfectant solution to fade.

In an embodiment, a reversibly protected colorant is added to and are stable in the solution comprising the disinfectant and the disinfectant solution remains uncolored. The reversibly protected colorant is configured to release the colorant from its protection after exposure to chemical or mechanical action on the disinfection solution. For example, the application of mechanical action depletes the imparted protection releasing the colorant, which releases the colorant and imparts color to the disinfectant solution, wherein the color of the disinfectant solution fades after a period of time.

In an embodiment, a reversibly protected colorant and a fading agent are added to and are stable in the solution comprising the disinfectant and the disinfectant solution remains uncolored. The reversibly protected colorant is configured to release the colorant from its protection after exposure to chemical or mechanical action on the disinfection solution. For example, the application of mechanical action depletes the imparted protection releasing the colorant, which releases the colorant and imparts color to the disinfectant solution and exposes the colorant to the fading agent, wherein the fading agent degrades the colorant and causes the color of the disinfectant solution to fade after a period of time.

In another embodiment, a reversibly protected colorant and reversibly protected fading agent are added to and are stable in the solution comprising the disinfectant and the disinfectant solution remains uncolored. The reversibly protected colorant and reversibly protected fading agent are configured to release the colorant and fading agent from their protection after exposure to chemical or mechanical action on the colored disinfection solution. For example, the application of mechanical action depletes the imparted protection releasing both the colorant and fading agent, which imparts color to the disinfectant solution and exposes the colorant to the fading agent, wherein the fading agent degrades the colorant and causes the color of the disinfectant solution to fade after a period of time.

As used herein, the term “mechanical action” to remove the protection refers to any manner of applying a force to remove the protection imparted by the protective material or structural modification, such as wiping, rubbing, grinding, or shaking, and causes the removal of the protection from the protected species to be released from the protection. For example, release of the colorant from its protection exposes the colorant to the fading agent and/or conditions that result in the colorant permanently losing its color over a period of time.

As used herein, “chemical action” to remove the protection refers to the use of a reactive agent to degrade or rupture the protection, thereby releasing the reversibly protected colorant or reversibly protected fading agent (when present), or both, into the disinfectant solution. The reactive agent may be activated by solvation, enzymatic cleavage, exposure to air, exposure to radiation (including light or heat), interaction with a magnetic field, interaction with an electric field, interaction with the disinfectant solution itself, or changes to solution pH or temperature, and any combination thereof; to degrade or rupture the protection over a period of seconds to hours. In some embodiments, the reactive agent is stable in the disinfectant solution and degrades or ruptures the protection to expose the previously reversibly protected species upon activation.

In other embodiments, the reactive agent is stabilized as a delivery and release system, such as core-shell particles. For example, the core may include the reactive agent, separated from the protective shell using a phase change material, which changes upon reaching specific chemical conditions to release the reactive agent from the core and promote inside-out degradation of the protection. In some embodiments, reversibly protected colorant is included in the core with the reactive agent.

In embodiments, exposure of the colorant to the disinfectant solution causes the solution to lose its color. In embodiments, release of the colorant from its protection exposes the colorant to external conditions such as air, light, or change in pH, which causes the colorant to lose its color. In embodiments, exposure of the colorant to the fading agent causes the solution to lose its color. In embodiments, the colorant loses its color due to oxidation.

For purposes of the instant invention, oxidation of the colorant to remove its optical properties (i.e., color) is irreversible. In other words, after the colored disinfectant solution has been applied to the surface or object, and the colored disinfectant solution loses its color over a period of time, the color of the colorant cannot be reconstituted by changing the conditions on/around the surface or object. The permanent removing of the color prevents staining of the surface or object.

Additionally, the removal of color from the colored disinfectant solution is not a result of the washing away the disinfectant solution with bulk volume of water, for example as what happens during handwashing or the use laundry detergent to wash clothes.

The reversibly protected colorant and compositions thereof as described here, when mixed with an aqueous disinfectant solution, are sufficient to color the disinfectant solution resulting in a colored disinfectant solution. The reversibly protected colorant and compositions thereof are adapted to provide a duration of color to the colored disinfectant solution for a period of time to achieve majority color fading. As used herein, “a period of time” can be any suitable period of time ranging from about 30 seconds to about 30 minutes after the colorant solution is applied to a surface or object and the protection is removed. In embodiments, the period of time is selected from 30 seconds to 30 minutes, preferably from about 30 seconds to 3 minutes, 30 seconds to 5 minutes, 5 minutes to 10 minutes, or 10 minutes to 15 minutes. In embodiments, the period of time is selected from 60 seconds to 30 minutes, preferably from about 60 seconds to 3 minutes, or 60 seconds to 5 minutes. In embodiments, the period of time is about 30 seconds or about 1, 2, 3, 5, 8, 10, 12, 15, or 30 minutes. In embodiments, the period of time is the time required to completely cover a surface and disinfect the surface. The actual time of color duration may be adjusted by addition of dye stabilizers or destabilizers to the composition.

In this way the invention allows users to explicitly see where the disinfectant has been applied without permanently staining or coloring the surface or object to which it has been applied.

As used herein, a “disinfectant” generally refers to any solid or liquid agent that destroys, inactivates, or significantly reduces the concentration of pathogens, provided that the disinfectant is not soap, such as hand or body soap, or laundry detergent. Such pathogens include bacteria, fungus, or viruses.

In embodiments, disinfectant includes but is not limited to chlorine and chlorine-based compounds, alcohols, formaldehyde, glutaraldehyde, peroxide compounds, iodophors, peracetic acid, phenols, ammonia compounds, quaternary ammonium compounds, and mixtures thereof. In embodiments, the disinfectant is a liquid (e.g., aqueous solution) used to disinfect hard surfaces, such as chlorine and chlorine-based compounds, alcohols, peroxide compounds, peracetic acid, quaternary ammonium compounds, and mixtures thereof.

In embodiments, the disinfectant is selected from quaternary ammonium salts, bleaches, alcohols, peroxides, oxidants, natural agents, soaps, and surfactants. In some embodiments, the disinfect includes, but is not limited to, a hypochlorite based disinfectant, a hypochlorous add based disinfectant, a dichloroisocyanurate based disinfectant, such as sodium dichloroisocyanurate, a quaternary ammonium based disinfectant, a quaternary ammonium/alcohol based disinfectant, an alcohol based disinfectant, an acid/alkali based disinfectant, a heavy metal based disinfectant, an aldehyde based disinfectant, a peroxide based disinfectant, for example a hydrogen peroxide based disinfectant, or a peracetic acid based disinfectant.

In some implementations, the disinfectant composition is an aqueous solution comprising a disinfectant selected from, but not limited to, sodium hypochlorite, sodium dichloroisocyanurate, potassium dichloroisocyanurate, hypochlorous acid, hydrogen peroxide, ethyl alcohol, a quaternary ammonium compound, a mixture of a quaternary ammonium compounds, a mixture of a quaternary ammonium compound(s) and an alcohol(s), an alcohol, peracetic acid, accelerated hydrogen peroxide, chlorine dioxide, calcium hypochlorite, chlorhexidine gluconate, glutaraldehyde, formaldehyde, phenol, acids, such as citric acid, and botanicals and/or essential oils, such as thymol. In some implementations, the disinfectant composition may comprise aldehydes (e.g., formaldehyde, glutaraldehye, and ortho-phthalaldehyde), hydrogen peroxide-peracetic acid combinations, iodophors, and phenols or phenolics.

In some implementations, the disinfectant composition is an aqueous solution comprising a disinfectant selected from sodium hypochlorite, sodium dichloroisocyanurate, potassium dichloroisocyanurate, hypochtorous acid, hydrogen peroxide, ethyl alcohol, a quaternary ammonium compound, a mixture of a quaternary ammonium compounds, a mixture of a quaternary ammonium compound(s) and an alcohol(s), an alcohol, peracetic acid, accelerated hydrogen peroxide, chlorine dioxide, calcium hypochlorite, chlorhexidine gluconate, glutaraldehyde, formaldehyde, and phenol. In some implementations, the disinfectant composition may comprise aldehydes (e.g., formaldehyde, glutaraldehye, and ortho-phthalaldehyde), hydrogen peroxide-peracetic acid combinations, iodophors, and phenols or phenolics. Typical disinfectant solutions compatible with the compositions described here include aqueous solutions of common disinfecting agents, for example, sodium hypochlorite, calcium hypochlorite, sodium dichloroisocyanurate, hydrogen peroxide, chlorine dioxide, peracetic acid, quaternary ammonium chloride, and alcohols.

A “fading agent” as defined herein refers to as any chemical agent or condition that, when combined with the colorant, causes the colorant to permanently change from colored to colorless. A fading agent includes, but is not limited to, a pH adjuster (e.g., an acid or a base), oxidizing agents, thermal-radical initiators, UV-radical indicators, light, bleaches, peroxide, solvents, compounds that promote or degrade supramolecular association or agglomeration, and combinations thereof.

In embodiments, the fading agent is a water-soluble oxidizing agent. In embodiments, the water-soluble oxidizing agent includes but is not limited to acids, organic peroxides, inorganic peroxides, persulfate compounds, hypochlorite compounds, chlorite compounds, chlorate compounds, perchlorate compounds, iodate compounds, dichromate compounds, lead dioxide, and permanganate compounds. In embodiments, the acid includes but is not limited to nitric acid, sulfuric acid, peroxydisulfuric acid, peroxymonosulfuric acid, and phosphoric acid. In embodiments, the oxidizing agent is a compound that decomposes into peroxy, chloroperoxy, hydroxy, perchlorate, chlorate or persulfate radicals. Such oxidizing agents include but are not limited to benzoyl peroxide, hydrogen peroxide, sodium peroxide, potassium peroxymonosulfate, sodium persulfate, sodium perborate, sodium chlorite, sodium chlorate, and their analogs or derivatives.

The invention provides a colorant that is protected to preserve the colorant's optical. properties until the application of mechanical or chemical action releases the colorant from its protection. The term “colorant”, as defined herein, include any dye, chromophore, or pigment that conveys optical absorption in the visible spectrum (300 to 700 nm) to the solution in which it is added. The colorant can be an organic compound that imparts visible color to an aqueous solution. A colorant can include any acid dye, reactive dye, basic dye, FD&C dye, solvent dye, pigment, chromophore, or light emitting chromophore. A colorant can also include an organic or organometallic molecule or compound including indicators that impart optical absorption between 300 and 700 nm in some media but not others, sensitive to, for example, pH, temperature, solvent system, gas exposure, light exposure.

In embodiments, the colorant is an oxidizable colorant. As used herein, an oxidizable colorant is a colorant that loses its color when oxidized. In embodiments, the loss of color due to oxidization is permanent.

In one embodiment, the colorant is an organic colorant. In embodiments, the organic colorant includes but is not limited to dyes derived from natural sources or synthetic molecules. In embodiments the natural dyes include but are not limited to anthocyanin dyes, anthraquinone dyes, azo dyes, azulene dyes, cyanine dyes. dioxazine dyes, xanthene dyes, indole dyes, indophenol dyes, indigoid dyes, napthol dyes, napthoxazine dyes, oxoindoline dyes, phenoxazine dyes, phthalein dyes, phthalocyanine dyes, nitrosulphonate dyes, pyrene dyes, thiazine dyes, thiophene dyes, triarylmethane dyes, quinoline dyes, and their derivatives, lakes, or mixtures thereof.

In embodiments, the colorant is an anthocyanin dye. In embodiments, the anthocyanin dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the anthocyanin dye is functionalized with one or more halogens, sulfonates, sulfates, amines, alkyl chains, alcohols, alkoxylates, phosphates, nitrates, carboxylates, or combinations thereof. In embodiments, the anthocyanin dye is functionalized with one or more halogens or sulfonates. In embodiments, the anthocyanin dye is functionalized with a sulfonate. In embodiments, the anthocyanin dye is halogenated. In embodiments, the colorant is a halogenated anthocyanin dye wherein the dye is functionalized with at least one halogen selected from chlorine, bromine, fluorine, iodine, astatine, and combinations thereof. In embodiments, the halogen is chlorine.

In embodiments, the colorant is an anthraquinone dye. In embodiments, the anthraquinone dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the anthraquinone dye is functionalized with one or more halogens, sulfonates, sulfates, amines, alkyl chains, alcohols, alkoxylates, phosphates, nitrates, carboxylates, or combinations thereof. In embodiments, the anthraquinone dye is functionalized with one or more halogens or sulfonates. In embodiments, the anthraquinone dye is functionalized with a sulfonate. In embodiments, the anthraquinone dye is halogenated. In embodiments, the colorant is a halogenated anthraquinone dye wherein the dye is functionalized with at least one halogen, selected from chlorine, bromine, fluorine, iodine, astatine, and combinations thereof. In embodiments, the halogen is chlorine.

In embodiments, the colorant is an azo dye. In embodiments, the azo dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the azo dye is functionalized with one or more halogens, sulfonates, sulfates, amines, alkyl chains, alcohols, alkoxylates, phosphates, nitrates, carboxylates, or combinations thereof. In embodiments, the azo dye is functionalized with one or more halogens or sulfonates. In embodiments, the azo dye is functionalized with a sulfonate. In embodiments, the azo dye is halogenated. In embodiments, the colorant is a halogenated azo dye wherein at the dye is functionalized with at least one halogen, selected from chlorine, bromine, fluorine, iodine, astatine, and combinations thereof. In embodiments, the halogen is chlorine.

In embodiments, the colorant is an azulene dye. In embodiments, the azulene dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the azulene dye is functionalized with one or more halogens, sulfonates, sulfates, amines, alkyl chains, alcohols, alkoxylates, phosphates, nitrates, carboxylates, or combinations thereof. In embodiments, the azulene dye is functionalized with one or more halogens or sulfonates. In embodiments, the azulene dye is functionalized with a sulfonate. In embodiments, the azulene dye is halogenated. In embodiments, the colorant is a halogenated azulene dye wherein the dye is functionalized with at least one halogen, selected from chlorine, bromine, fluorine, iodine, astatine, and combinations thereof. In embodiments, the halogen is chlorine.

In embodiments, the colorant is a cyanine dye. In embodiments, the cyanine dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the cyanine dye is functionalized with one or more halogens, sulfonates, sulfates, amines, alkyl chains, alcohols, alkoxylates, phosphates, nitrates, carboxylates, or combinations thereof. In embodiments, the cyanine dye is functionalized with one or more halogens or sulfonates. In embodiments, the cyanine dye is functionalized with a sulfonate. In embodiments, the cyanine dye is halogenated. In embodiments, the colorant is a halogenated cyanine dye wherein the dye is functionalized with at least one halogen, selected from chlorine, bromine, fluorine, iodine, astatine, and combinations thereof. In embodiments, the halogen is chlorine.

In embodiments, the colorant is a dioxazine dye. In embodiments, the dioxazine dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the dioxazine dye is functionalized with one or more halogens, sulfonates, sulfates, amines, alkyl chains, alcohols, alkoxylates, phosphates, nitrates, carboxylates, or combinations thereof. In embodiments, the dioxazine dye is functionalized with one or more halogens or sulfonates. In embodiments, the dioxazine dye is functionalized with a sulfonate. In embodiments, the dioxazine dye is halogenated. In embodiments, the colorant is a halogenated dioxazine dye wherein the dye is functionalized with at least one halogen, selected from chlorine, bromine, fluorine, iodine, astatine, and combinations thereof. In embodiments, the halogen is chlorine.

In embodiments, the colorant is a xanthene dye. In embodiments, the xanthene dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the xanthene dye is functionalized with one or more halogens, sulfonates, sulfates, amines, alkyl chains, alcohols, alkoxylates, phosphates, nitrates, carboxylates, or combinations thereof. In embodiments, the xanthene dye is functionalized with one or more halogens or sulfonates. In embodiments, the xanthene dye is functionalized with a sulfonate. In embodiments, the xanthene dye is halogenated. In embodiments, the colorant is a halogenated xanthene dye wherein the dye is functionalized with at least one halogen, selected from chlorine, bromine, fluorine, iodine, astatine, and combinations thereof. In embodiments, the halogen is chlorine.

In embodiments, the colorant is an indole dye. In embodiments, the indole dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the indole dye is functionalized with one or more halogens, sulfonates, sulfates, amines, alkyl chains, alcohols, alkoxylates, phosphates, nitrates, carboxylates, or combinations thereof. In embodiments, the indole dye is functionalized with one or more halogens or sulfonates. In embodiments, the indole dye is functionalized with a sulfonate. In embodiments, the indole dye is halogenated. In embodiments, the colorant is a halogenated indole dye wherein the dye is functionalized with at least one halogen, selected from chlorine, bromine, fluorine, iodine, astatine, and combinations thereof. In embodiments, the halogen is chlorine.

In embodiments, the colorant is an indophenol dye. In embodiments, the indophenol dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the indophenol dye is functionalized with one or more halogens, sulfonates, sulfates, amines, alkyl chains, alcohols, alkoxylates, phosphates, nitrates, carboxylates, or combinations thereof. In embodiments, the indophenol dye is functionalized with one or more halogens or sulfonates. In embodiments, the indophenol dye is functionalized with a sulfonate. In embodiments, the indophenol dye is halogenated. In embodiments, the colorant is a halogenated indophenol dye wherein the dye is functionalized with at least one halogen, selected from chlorine, bromine, fluorine, iodine, astatine, and combinations thereof. In embodiments, the halogen is chlorine.

In embodiments, the colorant is an indigoid dye. In embodiments, the indigoid dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the indigoid dye is functionalized with one or more halogens, sulfonates, sulfates, amines, alkyl chains, alcohols, alkoxylates, phosphates, nitrates, carboxylates, or combinations thereof. In embodiments, the indigoid dye is functionalized with one or more halogens or sulfonates. In embodiments, the indigoid dye is functionalized with a sulfonate. In embodiments, the indigoid dye is halogenated. In embodiments, the colorant is a halogenated indigoid dye wherein the dye is functionalized with at least one halogen, selected from chlorine, bromine, fluorine, iodine, astatine, and combinations thereof. In embodiments, the halogen is chlorine.

In embodiments, the colorant is a napthol dye. In embodiments, the napthol dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the napthol dye is functionalized with one or more halogens, sulfonates, sulfates, amities, alkyl chains, alcohols, alkoxylates, phosphates, nitrates, carboxylates, or combinations thereof. In embodiments, the napthol dye is functionalized with one or more halogens or sulfonates. In embodiments, the napthol dye is functionalized with a sulfonate. In embodiments, the napthol dye is halogenated. In embodiments, the colorant is a halogenated napthol dye wherein the dye is functionalized with at least one halogen, selected from chlorine, bromine, fluorine, iodine, astatine, and combinations thereof. In embodiments, the halogen is chlorine.

In embodiments, the colorant is a napthoxazine dye. In embodiments, the napthoxazine dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the napthoxazine dye is functionalized with one or more halogens, sulfonates, sulfates, amines, alkyl chains, alcohols, alkoxylates, phosphates, nitrates, carboxylates, or combinations thereof. In embodiments, the napthoxazine dye is functionalized with one or more halogens or sulfonates. In embodiments, the napthoxazine dye is functionalized with a sulfonate. In embodiments, the napthoxazine dye is halogenated. In embodiments, the colorant is a halogenated napthoxazine dye wherein the dye is functionalized with at least one halogen, selected from chlorine, bromine, fluorine, iodine, astatine, and combinations thereof. In embodiments, the halogen is chlorine.

In embodiments, the colorant is an oxoindoline dye. In embodiments, the oxoindoline dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the oxoindoline dye is functionalized with one or more halogens, sulfonates, sulfates, amines, alkyl chains, alcohols, alkoxylates, phosphates, nitrates, carboxylates, or combinations thereof. In embodiments, the oxoindoline dye is functionalized with one or more halogens or sulfonates. In embodiments, the oxoindoline dye is functionalized with a sulfonate. In embodiments, the oxoindoline dye is halogenated. In embodiments, the colorant is a halogenated oxoindoline dye wherein the dye is functionalized with at least one halogen, selected from chlorine, bromine, fluorine, iodine, astatine, and combinations thereof. In embodiments, the halogen is chlorine,

In embodiments, the colorant is a phenoxazine dye. In embodiments, the phenoxazine dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the phenoxazine dye is functionalized with one or more halogens, sulfonates, sulfates, amines, alkyl chains, alcohols, alkoxylates, phosphates, nitrates, carboxylates, or combinations thereof. In embodiments, the phenoxazine dye is functionalized with one or more halogens or sulfonates. In embodiments, the phenoxazine dye is functionalized with a sulfonate. In embodiments, the phenoxazine dye is halogenated. In embodiments, the colorant is a halogenated phenoxazine dye wherein the dye is functionalized with at least one halogen, selected from chlorine, bromine, fluorine, iodine, astatine, and combinations thereof. In embodiments, the halogen is chlorine.

In embodiments, the colorant is a phthalein dye. In embodiments, the phthalein dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the phthalein dye is functionalized with one or more halogens, sulfonates, sulfates, amines, alkyl chains, alcohols, alkoxylates, phosphates, nitrates, carboxylates, or combinations thereof. In embodiments, the phthalein dye is functionalized with one or more halogens or sulfonates. In embodiments, the phthalein dye is functionalized with a sulfonate. In embodiments, the phthalein dye is halogenated. In embodiments, the colorant is a halogenated phthalein dye wherein the dye is functionalized with at least one halogen, selected from chlorine, bromine, fluorine, iodine, astatine, and combinations thereof. In embodiments, the halogen is chlorine.

In embodiments, the colorant is a phthalocyanine dye. In embodiments, the phthalocyanine dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the phthalocyanine dye is functionalized with one or more halogens, sulfonates, sulfates, amines, alkyl chains, alcohols, alkoxylates, phosphates, nitrates, carboxylates, or combinations thereof. In embodiments, the phthalocyanine dye is functionalized with one or more halogens or sulfonates. In embodiments, the phthalocyanine dye is functionalized with a sulfonate. In embodiments, the phthalocyanine dye is halogenated. In embodiments, the colorant is a halogenated phthalocyanine dye wherein the dye is functionalized with at least one halogen, selected from chlorine, bromine, fluorine, iodine, astatine, and combinations thereof. In embodiments, the halogen is chlorine.

In embodiments, the colorant is a nitrosulphonate dye. In embodiments, the nitrosulphonate dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the nitrosulphonate dye is functionalized with one or more halogens, sulfonates, sulfates, amines, alkyl chains, alcohols, alkoxylates, phosphates, nitrates, carboxylates, or combinations thereof. In embodiments, the nitrosulphonate dye is functionalized with one or more halogens or sulfonates. In embodiments, the nitrosulphonate dye is functionalized with a sulfonate. In embodiments, the nitrosulphonate dye is halogenated. In embodiments, the colorant is a halogenated nitrosulphonate dye wherein the dye is functionalized with at least one halogen, selected from chlorine, bromine, fluorine, iodine, astatine, and combinations thereof. In embodiments, the halogen is chlorine.

In embodiments, the colorant is a pyrene dye. In embodiments, the pyrene dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the pyrene dye is functionalized with one or more halogens, sulfonates, sulfates, amines, alkyl chains, alcohols, alkoxylates, phosphates, nitrates, carboxylates, or combinations thereof. In embodiments, the pyrene dye is functionalized with one or more halogens or sulfonates. In embodiments, the pyrene dye is functionalized with a sulfonate. In embodiments, the pyrene dye is halogenated. In embodiments, the colorant is a halogenated pyrene dye wherein the dye is functionalized with at least one halogen, selected from chlorine, bromine, fluorine, iodine, astatine, and combinations thereof. In embodiments, the halogen is chlorine.

In embodiments, the colorant is a thiazine dye. In embodiments, the thiazine dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the thiazine dye is functionalized with one or more halogens, sulfonates, sulfates, amines, alkyl chains, alcohols, alkoxylates, phosphates, nitrates, carboxylates, or combinations thereof. In embodiments, the thiazine dye is functionalized with one or more halogens or sulfonates. In embodiments, the thiazine dye is functionalized with a sulfonate. In embodiments, the thiazine dye is halogenated. In embodiments, the colorant is a halogenated thiazine dye wherein the dye is functionalized with at least one halogen, selected from chlorine, bromine, fluorine, iodine, astatine, and combinations thereof. In embodiments, the halogen is chlorine.

In embodiments, the colorant is a thiophene dye. In embodiments, the thiophene dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the thiophene dye is functionalized with one or more halogens, sulfonates, sulfates, amines, alkyl chains, alcohols, alkoxylates, phosphates, nitrates, carboxylates, or combinations thereof. In embodiments, the thiophene dye is functionalized with one or more halogens or sulfonates. In embodiments, the thiophene dye is functionalized with a sulfonate. In embodiments, the thiophene dye is halogenated. In embodiments, the colorant is a halogenated thiophene dye wherein the dye is functionalized with at least one halogen, selected from chlorine, bromine, fluorine, iodine, astatine, and combinations thereof. In embodiments, the halogen is chlorine.

In embodiments, the colorant is a quinoline dye. In embodiments, the quinoline dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the quinoline dye is functionalized with one or more halogens, sulfonates, sulfates, amines, alkyl chains, alcohols, alkoxylates, phosphates, nitrates, carboxylates, or combinations thereof. In embodiments, the quinoline dye is functionalized with one or more halogens or sulfonates. In embodiments, the quinoline dye is functionalized with a sulfonate. In embodiments, the quinoline dye is halogenated. In embodiments, the colorant is a halogenated quinoline dye wherein the dye is functionalized with at least one halogen, selected from chlorine, bromine, fluorine, iodine, astatine, and combinations thereof. In embodiments, the halogen is chlorine.

In embodiments, the colorant is a triarylmethane dye. In embodiments, the triarylmethane dye includes solvent dyes, reactive dyes, basic dyes, direct dyes, mordant dyes, and acid dyes, which may be cationic, anionic, neutral, amphoteric, zwitterionic, or amphiphilic, combinations thereof. In embodiments, the quinoline dye is functionalized with one or more halogens, sulfonates, sulfates, amines, alkyl chains, alcohols, alkoxylates, phosphates, nitrates, carboxylates, or combinations thereof. In embodiments, the quinoline dye is functionalized with one or more halogens or sulfonates. In embodiments, the quinoline dye is functionalized with a sulfonate. In embodiments, the triarylmethane dye is halogenated. In embodiments, the colorant is a halogenated triarylmethane dye wherein the dye is functionalized with at least one halogen, selected from chlorine, bromine, fluorine, iodine, astatine, and combinations thereof. In embodiments, the halogen is chlorine.

In embodiments, the colorant is selected from Mordant Blue 1, Basic Blue 1, Acid Green 9, CI Pigment Blue 9, Alkali Fast Green 10GA, ethanaminium N-[4-[(2-chlorophenyl)[4-[(2-cyanoethypethylamino]phenyl]methylene]-2,5-cyclohexadien-1-ylidene]-2-cyano-N-ethyl-, chloride (1:1), hydrogen [4-[(2-chlorophenyll)[4-[ethyl(sulphonatobenzyl)amino]phenyl]methylene]cyclohexa-2,5-dien-1-ylidene]ethyl)(sulphonatobenzyl) ammonium, and their alkoxylated, substituted, sulfonated, and polymeric derivatives.

The terms “FD&C” and “D&C” dyes are recognized in the art. In the United States, colorants for food, drugs and cosmetics are regarded as “color additives”. The Federal Food Drug & Cosmetic (FD&C) Act of 1938 made food color additive certification mandatory. Since then, the Food and Drug Administration (FDA) has been responsible for regulating all color additives used in food, drugs and cosmetics. Each batch to be sold in the United States has to be certified by the FDA. To avoid confusing color additives used in food with those manufactured for other uses, 3 categories of certifiable color additives were created: 1) FD&C (Food, Drug & Cosmetics) color additives with applications in food, drug & cosmetics; 2) D&C (Drug & Cosmetics) color additives with applications in drug & cosmetics; 3) External D&C (External Drug & Cosmetics) color additives with applications in externally applied drugs & in externally applied cosmetics. The use of all food colors approved for use in the United States are listed in 21 CFR (Code of Federal Regulation), parts 70 through 82 dealing with color additives.

Representative examples of basic dyes useful in the present compositions include, but are not limited to: Basic Black 2, Basic Blue 1, Basic Blue 3, Basic Blue 6, Basic Blue 7, Basic Blue 9, Basic Blue 11, Basic Blue 12, Basic Blue 16, Basic Blue 17, Basic Blue 24, Basic Blue 26, Basic Blue 41, Basic Blue 66, Basic Blue 140, Basic Brown 1, Basic Brown 4, Basic fuchsin, Basic Green 1, Basic Green 4, Basic Green 5, Basic Orange 2, Basic Orange 14, Basic Orange 21, Basic Red 1, Basic Red 2, Basic Red 5, Basic Red 9, Basic Red 29, Basic Violet 1, Basic Violet 2, Basic Violet 3, Basic Violet 4, Basic Violet 10, Basic Yellow 1, Basic Yellow 2, and mixtures thereof.

Representative examples of FD&C dyes useful in compositions of the invention include, but are not limited to: FD&C Blue 1, FD&C Blue 2, FD&C Green 3, FD&C Red 3, FD&C Red 40, FD&C Yellow 5, FD&C Yellow 6, Fast Emerald Green, and mixtures thereof.

As used herein, the terms “reversibly protected” refers to any colorant and/or fading agent that is modified, coated, encapsulated, and/or combined with other compounds to create a composite molecule or material that preserves the colorant's optical properties or limits the fading agent's activity until the application of a chemical or mechanical action that degrades the protection. Protection mediates the interaction of the colorant with a fading agent to preserve the optical properties of the colorant until mechanical action modifies or removes the protection. Upon release, the colorant is exposed to the disinfectant solution and/or a fading agent, if present, and/or conditions***, which results in a permanent loss of the visible color from the colorant.

In embodiments, the reversibly protected species is a colorant or fading agent that is fully or partially encapsulated, fully or partially coated, fully or partially shielded, or structurally modified. In embodiments, the reversibly protected colorant is a colorant that is fully or partially encapsulated, fully or partially coated, fully or partially shielded, or structurally modified. In embodiments, the reversibly protected fading agent is a fading agent that is fully or partially encapsulated, fully or partially coated, fully or partially shielded, or structurally modified.

In some embodiments, the reversibly protected colorant of the invention is an encapsulated colorant or a colorant within a core shell structure. The encapsulated colorant or core shell structure protects the colorant's optical properties until mechanical action exposes the colorant, for example, by releasing the colorant from the encapsulation or core shell structure.

In embodiments where the protection is imparted by encapsulation or coating, this can be accomplished by encapsulating or coating, partially or fully, the colorant or fading agent with a separate material including but not limited to waxes, sugars, polymers, lipids, and inorganic shells such as metals, semimetals, non-metals, and metal oxides.

In some embodiments, the reversibly protected colorant is a colorant that is encapsulated. In some embodiments the colorant is partially encapsulated. In some embodiments the colorant is fully encapsulated. In embodiments, the reversibly protected colorant is a colorant encapsulated in one or more polymers, polyelectrolytes; resins; animal proteins; plant proteins; inorganic shells; or any combination thereof. In embodiments, the one or more polymers is selected from polyvinyl alcohol), or cross linked polymers. In embodiments, the one or more polymers includes but is not limited to a polymer selected from methacrylate polymers, amine methacrylate polymers, poly-acrylic acid, poly-methacrylic acid, copolymers of poly-acrylic acid, copolymers of poly-methacrylic acid, styrene-(meth)acrylates, maleic acid, polyvinylacetates, vinyl acrylic copolymers, vinyl methacrylic copolymers, silicone polymers, polyureathane polymers, melamine formaldehyde systems, coacervates of polyelectrolytes, or combinations thereof. In embodiments, the one or more polyelectrolytes is selected from poly(acrylic acid), or poly(diallyldimethylammonium chloride. In embodiments, the one or more resins is selected from melamine, urea formaldehyde, or polyurethane, In embodiments, the one or more animal proteins is selected from whey, gelatin, albumin, or silk fibroin. In embodiments, the one or more plant proteins is selected from chitosan, alginate, gum arabic, pectin, carrageenan, cellulose, agar, waxes, or silane coupling agents. In embodiments, the one or more inorganic shells is selected from mica, titanium oxides, zinc oxides, silicates.

In embodiments, the reversibly protected colorant is a colorant that is fully coated or partially coated. In embodiments, the coating is selected from waxes, sugars, polymers, lipids, and inorganic shells such as metals, semimetals, non-metals, and metal oxides.

In some embodiments, the reversibly protected fading agent is a fading agent that is encapsulated. In some embodiments the fading agent is partially encapsulated. In some embodiments the fading agent is fully encapsulated. In embodiments, the reversibly protected fading agent is a fading agent encapsulated in one or more polymers, polyelectrolytes; resins; animal proteins; plant proteins; inorganic shells; or any combination thereof. In embodiments, the one or more polymers is selected from polyvinyl alcohol), or cross linked polymers. In embodiments, the one or more polymers includes but is not limited to a polymer selected from methacrylate polymers, amine methacrylate polymers, poly-acrylic acid, poly-methacrylic acid, copolymers of poly-acrylic acid, copolymers of poly-methacrylic, acid, styrene-(meth)acrylates, maleic acid, polyvinylacetates, vinyl acrylic copolymers, vinyl methacrylic copolymers, silicone polymers, polyureathane polymers, melamine formaldehyde systems, coacervates of polyelectrolytes, or combinations thereof. In embodiments, the one or more polyelectrolytes is selected from poly(acrylic acid), or poly(diallyldimethylammonium chloride. In embodiments, the one or more resins is selected from melamine, urea formaldehyde, or polyurethane. In embodiments, the one or more animal proteins is selected from whey, gelatin, albumin, or silk fibroin. In embodiments, the one or more plant proteins is selected from chitosan, alginate, gum arabic, pectin, carrageenan, cellulose, agar, waxes, or silane coupling agents. In embodiments, the one or more inorganic shells is selected from mica, titanium oxides, zinc oxides, silicates.

In embodiments, the reversibly protected fading agent is a fading agent that is fully coated or partially coated. In embodiments, the coating is selected from waxes, sugars, polymers, lipids, and inorganic shells such as metals, semimetals, nonmetals, and metal oxides.

In some embodiments, the colorant or fading agent is structurally modified. Structural modifications include, but are not limited to, intrinsic or extrinsic spacers or steric protection.

Other modes of protection such as shielding the electronic system from which the optical absorption or fading activity derives may also be applied. In embodiments, the reversibly protected colorant is a colorant that is fully or partially shielded. In embodiments, the reversibly protected colorant is a colorant that is fully shielded. In embodiments, the reversibly protected colorant is a colorant that is partially shielded. In embodiments, the reversibly protected fading agent is a fading agent that is fully or partially shielded. In embodiments, the reversibly protected fading agent is a fading agent that is fully shielded. In embodiments, the reversibly protected fading agent is a fading agent that is partially shielded. Such shielding includes, but is not limited to, formation of discrete micelles, or modification of solvent/solvation to induce aggregation or assembly.

The composite material is fabricated by any method known in the art. Such methods include, but are not limited to coacervation, in situ polymerization, interfacial polymerization, matrix polymerization, cross-linking, spray drying, solvent evaporation, supercritical anti-solvent techniques, emulsion, and extrusion techniques.

In another aspect, the invention generally relates to a container comprising the reversibly protected species or a composition thereof as disclosed herein. In embodiments, the container comprises the reversibly protected colorant or a composition thereof In embodiments, the container comprises the reversibly protected fading agent or a composition thereof. The container can be any suitable receptacle, such as a packet, vial, carton, can, or jar, in which material is held or carried. In embodiments, the container is a packet.

In yet another aspect, the invention generally relates to a kit for coloring a disinfectant solution including one or more containers. In embodiments, the invention provides a kit for coloring a disinfectant solution wherein the kit comprises one or more containers comprising a reversibly protected colorant; optionally one or more containers comprising a fading agent; and instructions for combining the reversibly protected colorant with a disinfectant solution, alone or in combination with the fading agent; and wherein, when combined, the reversibly protected colorant is sufficient to color the disinfectant solution. In some embodiments, the fading agent is a reversibly protected fading agent. In embodiments, the components of the kit are adapted to be combined immediately prior to use. In some embodiments, the reversibly protected colorant is adapted to be combined with a disinfectant solution at the point of manufacture of the disinfectant solution and stored in unified packaging.

In embodiments, the invention provides a method for disinfecting a surface or an object In embodiments, the method comprises providing a disinfectant solution and a composition comprising (i) a reversibly protected colorant; (ii) a colorant and a reversibly protected fading agent; or (iii) a reversibly protected colorant and a reversibly protected fading agent; and combining an amount of composition (i), (ii), or (iii) and the disinfectant solution at or prior to the point of use, wherein the composition is sufficient to color the disinfectant solution; and applying the colored disinfectant solution to the surface or object; wherein, upon applying, the color of the disinfectant solution clearly marks where the spray or film has or has not been applied to the surface or object; and wherein colored disinfectant solution is subjected to a. chemical and/or mechanical action that releases the protected species (i.e., the reversibly protected colorant, the reversibly protected fading agent, or both) from the protection such that the color of the disinfectant solution fades to clear within a period of time.

In embodiments, the disinfectant solution and the composition are combined immediately prior to use. In embodiments, the disinfectant solution and the composition combined upon application to a surface or object to be disinfected.

In embodiments, the invention provides a method for disinfecting a surface or an object. In embodiments, the method comprises applying, to the surface or object, a colored disinfectant solution comprising a disinfectant and a reversibly protected colorant; wherein the reversibly protected colorant is sufficient to color the disinfectant solution; wherein, upon applying, the color of the disinfectant solution clearly marks where the spray or film has or has not been applied to the surface or object; and wherein disinfectant solution is subjected to a chemical and/or mechanical action that releases the colorant from the protection such that the color of the disinfectant solution fades to clear within a period of time. In embodiments, the colored disinfectant agent further comprises fading agent. In embodiments, the fading agent is a reversibly protected fading agent.

In embodiments, the invention provides a method for disinfecting a surface or an object wherein the method comprises applying, to the surface or object, a colored disinfectant solution comprising a disinfectant, a colorant and a reversibly protected fading agent; wherein the colorant is sufficient to color the disinfectant solution; wherein, upon applying, the color of the disinfectant solution clearly marks where the spray or film has or has not been applied to the surface or object; and wherein disinfectant solution is subjected to a chemical and/or mechanical action that releases the fading agent from the protection such that the color of the disinfectant solution fades to clear within a period of time.

In any of the methods disclosed herein, the surface or object is not a body part, such as hands, arms or skin.

In any of the methods disclosed herein, the disinfectant solution is any disinfectant solution as disclosed herein.

In any of the methods disclosed herein, the colored disinfectant solution is applied to the surface as a spray or film.

In any of the methods disclosed herein, the disinfectant solution is held within a wipe and the reversibly protected colorant or composition thereof is applied to the wipe. In embodiments, the colored disinfectant solution is applied to, or in the form of, a wipe, wherein the surface or object is wiped with the wipe.

In any of the methods disclosed herein, the concentration of the colorant, excluding the protection, in colored disinfectant solution is between 0.03 mMol-150 mMol. In any of the methods disclosed herein, the concentration of the colorant the in colored disinfectant is between 0.3 mMol-15 mMol. As used herein, the concentration of the colorant the in colored disinfectant solution refers to the concentration of the colorant after being mixed with disinfectant solution.

### EXAMPLES

### Methods and Data

### Complex Coacervation Microencapsulation of Thymolphthalein.

In some embodiments, components will be encapsulated by the techniques listed above. One such embodiment involves the process of complex coacervation microencapsulation. Complex coacervation microencapsulation involves phase separation of two immiscible liquid phases, resulting in a dilute (equilibrium) phase, and a dense (coacervate) phase characterized by a relatively high concentration of macromolecules. Coacervate macromolecules generally take the form of core-shell particles, wherein shell formation or deposition around the core material is driven primarily by electrostatic interactions between two, oppositely charged polymers (e.g., proteins or polysaccharides).

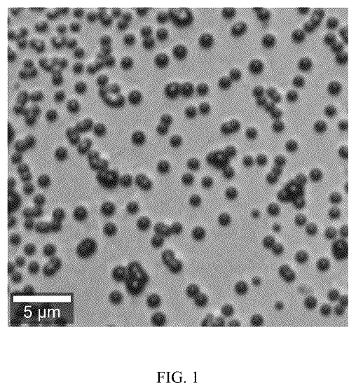

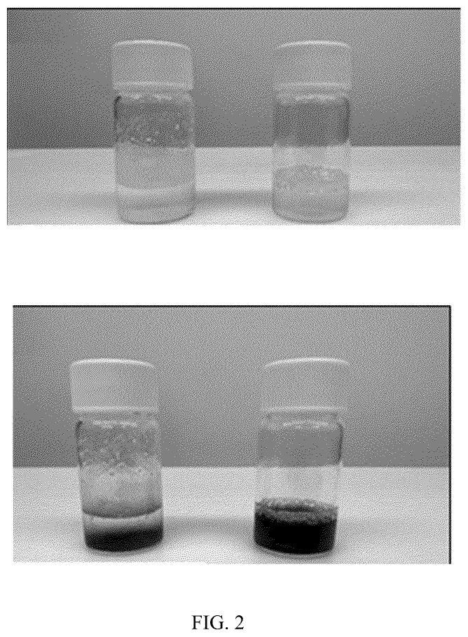

In one embodiment, the core suspension is first prepared by dispersing a water-soluble colorant (thymolphthalein, 0.107 g) in a polysorbate-type non-ionic surfactant (Tween 80, 5 g), then dispersed into chitosan solution (1% w/w acetic acid) under mechanical stirring at 50C until fully homogenized. Gelatin solution (1% w/w acetic acid) is added to the dye/chitosan mixture at a rate of 1 mL/minute under mechanical stirring at 50° C. for additional 30 minutes or until fully homogenized, Coacervation can be induced by adjusting the pH of the reaction mixture. The pH is adjusted to 5.5 by dropwise addition of 1M sodium hydroxide solution and stirred homogeneously at 50C for four hours, then gradually returned to room temperature under constant stirring. Finally, the liquid coacervate solution could be stirred under ice for one hour before isolating and drying. The thymolphthalein-loaded microcapsules can be isolated by centrifugation and washing with cold ethanol and deionized water, then freeze-dried in vacuo. Gelatin-chitosan microcapsulates loaded with thymolphthalein are shown in FIG. 1.

In some embodiments, microcapsules can be prepared using coacervation processes yielding a complex structure in which there is a large central core of encapsulated material.

Complex microcapsules can be prepared according to the following generic process. Gelatin is dissolved in deionized water in beakers that serve as the main reaction vessels. Gum arabic is dissolved into the deionized water. A conventional colorant composition is emulsified with a laboratory mixer to form a fine emulsion. The same colorant is then emulsified into the previously formed fine emulsion at a lower rpm such that after about 10 minutes a new, second, size distribution of emulsified “particles” with a mean size of about 100 microns (coarse emulsion) are produced. The fine emulsion is still present. The pH's are selected by observing the at which the coacervates start forming. The solution/emulsions are cooled to room temperature and allowed to stand for about 30 minutes. The coacervate is then cross-linked with a 25% solution of glutaraldehyde.

Chitosan microcapsules can be prepared from water-in-oil emulsions: Chitosan (>85% deacetylated, Aldrich 417963), MilliO water, Glacial acetic acid, FD&C blue #1 food coloring dye, Cetylpyridinium chloride (CPC), Mineral oil (white, heavy), and Sorbitan monooleate (Span-80) surfactant. A chitosan solution is prepared by mixing chitosan with water/1% (vol.) acetic acid solution. The chitosan is dissolved in 600 mL of water/1% (vol.) acetic acid solution by stirring and heating the mixture to approximately 60° C. for about 1 day. Then 3.3 mL of aqueous solution containing colorant (pre-dissolved) is added to 200 mL of the chitosan solution. A mixture is then prepared by adding an emulsifier (IKA RE162/P) with 800 mL of mineral oil and 8 mL of Span-80, and stirred at 100 rpm for 5 minutes to mix. The mixture is allowed to sit for 20 minutes to allow air bubbles to rise and pop. The chitosan solution is then added to the mixture through two 60 mL syringes with 16 gauge needles over a period of approximately 20 minutes. The resulting emulsion is then stirred for another 30 minutes after adding the last of 10 the chitosan solution. The emulsion is then transferred to a 2 L Pyrex beaker on a magnetic stir plate. The emulsion is heated to approximately 70° C. while stirring and left overnight (approximately 14-16 hours) to evaporate the acetic acid and most of 15 the water. The emulsion is then heated to approximately 95°-100° C. with continued stirring until the following day (approximately 24 hours), The emulsion is then collected into 50 mL centrifuge tubes and spun each at 3,500 rpm for 10 minutes. Most of the particles settle following centrifugation, but a large fraction of the smallest particles (having a size of a few microns or less) may remain suspended. The solution is decanted into waste. In half of the centrifuge tubes, particles are re-suspended in the remaining mineral oil using a glass pipette. All of the remaining particle-oil slurry is transferred from these tubes to a glass vial (total volume approximately 15 mL), and centrifuged again at 3,500 rpm for 1 hour. The solution is again decanted into waste and residual oil is removed with a pipette as far as possible. In the other half of the tubes, particles are resuspended in approximately 10 mL hexane in each tube by vortexing. Liquid is collected from all tubes into 2 tubes, which are then centrifuged at 3,500 rpm for 10 minutes. Solution is decanted into waste and particles are resuspended in approximately 10 mL, hexane in each tube. Finally, the solution is filtered through qualitative filter paper by gravity filtration and left to dry in a fume hood.

In some embodiments, microcapsules comprising an organic liquid fill enclosed by an impermeable shell of urea formaldehyde polymer can be prepared. These microcapsules are produced by dispersing and maintaining the fill material as finely divided particles in an aqueous, water-soluble urea formaldehyde precondensate solution.

A prepolymer solution is formed by heating a mixture of 488.5 grams (6.0 moles) 37% aqueous formaldehyde and 240 grams (4.0 moles) urea adjusted to pH 8.0 with triethanolamine for one hour at 70° C. This prepolymer is diluted with 1,000 grams of water, yielding a relatively , stable solution for forming microcapsules. Microcapsules containing dye solution are prepared by first adjusting the pH of one tenth volume of the above prepolymer solution with 10% aqueous citric acid while agitating, adding colorant at room temperature and further adjusting the pH with 10% citric acid, maintaining agitation sufficient to disperse the phases. The agitated mixture is heated and maintained at 40-45 C. After about one-half hour, shell formation is microscopically evident. The mixture thickens during 40 minutes at this elevated temperature, therefore 50 mL of warm water is added. After three hours reaction time, cold water is added to bring the total volume to 600 mL and one-half of the resulting slurry is filtered by gravity, yielding microcapsules after air drying. The remaining suspension is gravity filtered after standing 17 hours and air dried to yield additional microcapsules. The microcapsules are free flowing.

In some embodiments, the present invention provides substantially non-leachable sol-gel micro and nanoparticles encapsulating one or more agents (e.g., colorants). These particles are characterized by stability to leaching or migration of the agents from therein.

The water-soluble dye, for example, FD&C Blue No. 1 (0.002-0.2 gram), and Polysorbate 80 (0.01-1 gram) are dissolved in a solution of 30-70% acetic acid in water (10-40 grams). 10-40 grams tetraethyl orthosilicate (TEOS) are then added and the obtained solution is stirred at room temperature. The solution is emulsified in a stirred, cooled oil phase containing 5-15 grams sorbitan oleate and 100-300 grams castor oil. The obtained emulsion is poured into 100-800 grams decanol and the obtained mixture is stirred using a mechanical stirrer. A fine particulate powder is thus obtained and is collected by sedimentation in a centrifuge. The precipitate is washed consecutively with hexane, ethanol and hexane, and is dried in an oven. Then, 1-10 grams of the dry powder obtained are suspended in 40 grams TEOS and the mixture is stirred at room temperature. 0.01-5 N HCl (50-500 ul) is then added to the stirred dispersion. The obtained powder is separated, and the precipitate is consecutively washed with hexane, ethanol, NaOH-containing ethanol, sodium lauryl sulfate solution (SLS), water and ethanol. The obtained particles are then dried in an oven.

The reversibly protected species is further shown as a composition comprising water in oil, and oil in water microcapsules. Microcapsules are obtained through either oil in water (O/W) or water in oil (W/O) emulsifications. In one embodiment, microcapsules are obtained by steps comprising dispersing an oil soluble amine modified polyfunctional polyvinyl monomer (or oligomer) and an oil soluble bi- or poly functional vinyl monomer or oligomer along with a free radical initiator such as an azo or peroxy initiator and an organic acid into an internal phase oil which is a non-solvent for the water phase. The phase in excess is water with O/W emulsification. With W/O emulsifications the phase in excess or continuous phase is oil. The term internal phase oil is used for convenience and simplicity to refer to the oil phase and to refer to the type of oils conventionally used as the internal phase or contents of microcapsules in conventional microencapsulation. With the W/O emulsifications, the oil however ends up being the continuous phase. The water phase forms the capsule internal contents.

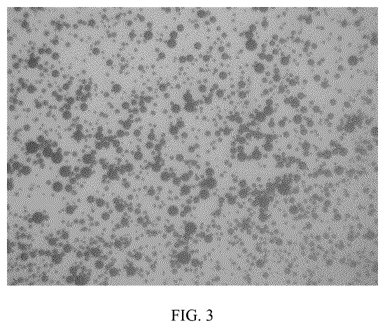

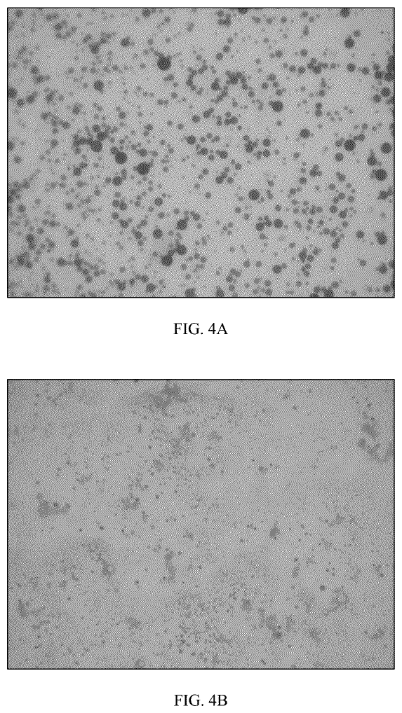

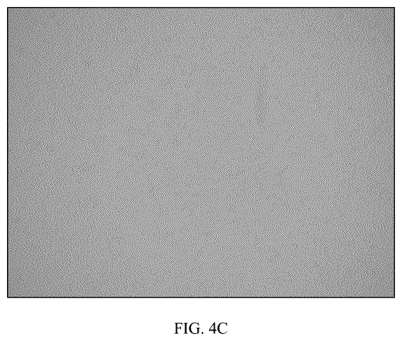

The term “oil phase” is intended to refer to the oil phase oil. The oil phase dispersion is heated for a time and temperature sufficient to oligomerize the amine modified polyfunctional polyvinyl monomer or oligomer and oil soluble bi- or polyfunctional vinyl monomer or oligomer forming a pre-polymer. Then a water phase comprising a dispersion in water of an emulsifier and an optional second initiator which can be the same or different such as an azo or peroxy initiator is added to the oil phase. This water phase is emulsified into the oil phase (W/O) followed by heating for a time and temperature sufficient to decompose at least one of the free radical initiators, which can be placed in either or both of the oil and/or water phases; thereby forming microcapsule wall material at the interface of the water and oil phases. A third heating step is used to polymerize the formed wall material and in the process, preferably decomposing any remaining initiator. Results are shown in FIG. 3 and FIGS. 4A and 4B.

While this invention has been particularly shown and described with references to preferred embodiments thereof, it will be understood by those skilled in the art that various changes in form and details may be made therein without departing from the scope of the invention encompassed by the appended claims.

1. A reversibly protected colorant which when mixed with a disinfectant solution imparts color to the disinfectant solution allowing for temporary visualization of a disinfectant, wherein the colorant is selected from anthocyanin dyes, anthraquinone dyes, azo dyes, azulene dyes, cyanine dyes, dioxazine dyes, xanthene dyes, indole dyes, indophenol dyes, indigoid dyes, napthol dyes, napthoxazine dyes, oxoindoline dyes, phenoxazine dyes, phthalein dyes, phthalocyanine dyes, nitrosulphonate dyes, pyrene dyes, thiazine dyes, thiophene dyes, triarylmethane dyes, quinoline dyes, and their derivatives, lakes, or mixtures thereof.

2. The reversibly protected colorant of claim 1, wherein the colorant is functionalized with one or more halogens, sulfonates, sulfates, amines, alkyl chains, alcohols, alkoxylates, phosphates, nitrates, carboxylates, or combinations thereof.

3. The reversibly protected colorant of claim 2, wherein the colorant is functionalized with one or more halogens or sulfonates.

4. The reversibly protected colorant of claim 2, wherein the colorant is halogenated.

5. The reversibly protected colorant of claim 1, wherein the colorant is fully or partially encapsulated, fully or partially coated, fully or partially shielded, or structurally modified.

6. The reversibly protected colorant of claim 5, wherein the colorant is fully or partially encapsulated.

7. The reversibly protected colorant of claim 6, wherein the colorant is encapsulated in one or more polymers, polyelectrolytes; resins; animal proteins; plant proteins; inorganic shells; or any combination thereof.

8. The reversibly protected colorant of claim 7, wherein the one or more polymers is selected from polyvinyl alcohol), or cross linked polymers.

9. The reversibly protected colorant of claim 7, wherein the one or more polyelectrolytes is selected from poly(acrylic acid), or poly(diallyldimethylammonium chloride.

10. The reversibly protected colorant of claim 7, wherein the one or more resins is selected from melamine, urea formaldehyde, or polyurethane.

11. The reversibly protected colorant of claim 7, wherein the one or more animal proteins is selected from whey, gelatin, albumin, or silk fibroin.

12. The reversibly protected colorant of claim 7, wherein the one or more plant proteins is selected from chitosan, alginate, gum arabic, pectin, carrageenan, cellulose, agar, waxes, or silane coupling agents.

13. The reversibly protected colorant of claim 7, wherein the one or more inorganic shells is selected from mica, titanium oxides, zinc oxides, silicates.

14. The reversibly protected colorant of claim 5, wherein the colorant is fully coated or partially coated.

15. The reversibly protected colorant of claim 14, wherein the coating is selected from waxes, sugars, polymers, lipids, and inorganic shells such as metals, semimetals, non-metals, and metal oxides.

16. The reversibly protected colorant of claim 5, wherein the colorant is structurally modified.

17. The reversibly protected colorant of claim 16, wherein the structural modification is selected from intrinsic or extrinsic spacers or steric protection.

18. The reversibly protected colorant of claim 5, wherein the colorant is fully or partially shielded.

19. The reversibly protected colorant of claim 18, wherein shielding is selected from discrete micelles, or modification of solvent/solvation to induce aggregation or assembly.

20. The reversibly protected colorant of claim 1, wherein the reversibly protected colorant is free of any surfactants, hydrotropes, thickening agents, or alkaline builders.

21. A composition comprising a reversibly protected colorant of claim 1.

22. The composition of claim 21, wherein the composition is a solid or liquid composition.

23. A colored disinfectant composition comprising a reversibly protected colorant and a disinfectant solution, wherein the reversibly protected colorant is sufficient to color the composition allowing for temporary visualization of a disinfectant.

24. The colored disinfectant composition of claim 23, further comprising a fading agent.

25. The colored disinfectant composition of claim 24, wherein the fading agent is a reversibly protected fading agent.

26. A colored disinfectant composition comprising a reversibly protected fading agent, a colorant and a disinfectant solution, wherein the colorant is sufficient to color the composition al lowing for temporary visualization of a disinfectant.

27. The colored disinfectant composition of claim 23, wherein the reversibly protect colorant is the reversibly protect colorant of any one of claims 1 to 20 or a composition thereof.

28. (canceled)

29. The colored disinfectant composition of claim 23, wherein the colored disinfectant composition is a disinfectant article.

30. A kit for a colored disinfectant solution comprising:
(i) one or more containers comprising a reversibly protected colorant;
(ii) optionally one or more containers comprising a fading agent; and
(iii) instructions for combining the reversibly protected colorant with a disinfectant solution, alone or in combination with the fading agent; wherein, when combined, the reversibly protected colorant is sufficient to color the disinfectant solution.

31. The kit according to claim 30, wherein the fading agent is a reversibly protected fading agent.

32. The kit according to claim 30, wherein the components are adapted to be combined immediately prior to use.

33. The kit according to claim 30, wherein the reversibly protected colorant is adapted to be combined with a disinfectant solution at the point of manufacture of the disinfectant solution and stored in unified packaging.

34. The kit of claim 30, wherein the reversibly protect colorant is the reversibly protect colorant of any one of claims 1 to 20 or a composition thereof.

35. A method for disinfecting a surface or an object, comprising providing:
a) a disinfectant solution; and
b) a composition comprising (i) a reversibly protected colorant; (ii) a colorant and a reversibly protected fading agent; or (iii) a reversibly protected colorant and a reversibly protected fading agent; and
c) combining an amount b)(i), b)(ii), or b)(iii) and the disinfectant solution, wherein the composition is sufficient to color the disinfectant solution; and
d) applying the colored disinfectant solution to the surface or object;
wherein, upon applying, the color of the disinfectant solution clearly marks where the colored disinfectant solution has or has not been applied to the surface or object; and wherein colored disinfectant solution is subjected to a chemical and/or mechanical action that releases the protected species from the protection such that the color of the disinfectant solution fades to clear within a period of time.

36. The method according to claim 35, wherein the components are combined immediately prior to use.

37. The method according to claim 35, wherein the components are combined upon application to a surface or object to be disinfected.

38. A method for disinfecting a surface or an object, comprising applying, to the surface or object, a disinfectant solution comprising a disinfectant and a reversibly protected colorant; wherein the reversibly protected colorant is sufficient to color the disinfectant solution; herein, upon applying, the color of the disinfectant solution clearly marks where the spray or film has or has not been applied to the surface or object; and wherein disinfectant solution is subjected to a chemical and/or mechanical action that releases the colorant from the protection such that the color of the disinfectant solution fades to clear within a period of time.

39. The method of claim 35, wherein the reversibly protect colorant is the reversibly protect colorant of any one of claims 1 to 20 or a corn position thereof.

40. A method for disinfecting a surface or an object, comprising applying, to the surface or object, a disinfectant solution comprising a disinfectant, a colorant and a reversibly protected fading agent; wherein the colorant is sufficient to color the disinfectant solution;
wherein, upon applying, the color of the disinfectant solution clearly marks where the spray or film has or has not been applied to the surface or object; and wherein disinfectant solution is subjected to a chemical and/or mechanical action that releases the fading agent from the protection such that the color of the disinfectant solution fades to clear within a period of time.

41. The method of claim 35, wherein the colored disinfectant solution is applied to the surface as a spray or film.

42. The method of claim 35, wherein the colored disinfectant solution is applied to, or in the form of, a disinfectant article, wherein the surface or object is wiped with the disinfectant article.

43. The method of claim 35, wherein the concentration of the colorant the in colored disinfectant is between 0.03 mMol and 150 mMol.

44. The method of claim 43, wherein the concentration of the colorant the in colored disinfectant is between 0.3 mMol and 15 mMol.
